(19) United States
(12) Patent Application Publication
(10) Pub. No.: US 2026/0364164 A1
(43) Pub. Date: Nov. 19, 2026

(54) MUCOSAL DRUG DELIVERY PREPARATIONS AND PREPARATION METHODS AND APPLICATIONS THEREOF

(71) Applicants: Beijing Abace Biotechnology Co., Ltd, Beijing (CN); ZW Pharm (Beijing) Co., Ltd., Beijing (CN)

(72) Inventors: Wenyi ZHANG, Beiijing (CN); Han CHEN, Beiijing (CN); Yuqi CAO, Beiijing (CN); Nan LI, Beiijing (CN); Changyou ZHAO, Beiijing (CN); Qingrui HUANG, Beiijing (CN); Shuo LIU, Beiijing (CN); Xiaofang LI, Beiijing (CN)

(21) Appl. No.: 19/122,796

(22) PCT Filed: Oct. 20, 2023
(86) PCT No.: PCT/CN2023/125644

§ 371 Date: Apr. 21, 2025

(30) Foreign Application Priority Data
Oct. 21, 2022 (CN) 202211299473.7
Oct. 11, 2023 (CN) 202311315364.4

(51) Int. Cl. A61K 39/215; A61K 9/00; A61K 9/51; A61K 39/00

(52) U.S. Cl. CPC A61K 39/215; A61K 9/0043; A61K 9/5123; A61K 9/5161; A61K 9/5192; A61K 2039/53; A61K 2039/543; C12N 2770/20034

(57) ABSTRACT

A mucosal administration formulation, a preparation method and use thereof. The preparation method of the mucosal administration formulation includes the following steps: (1) mixing a nucleating agent solution, a drug protein solution, and an inorganic salt buffer solution to obtain a mixed solution; (2) spraying the mixed solution obtained in step (1) into a coating agent solution under stirring, adding a permeation enhancer, and freeze-drying to obtain the formulation.

### CROSS-REFERENCE TO RELATED APPLICATIONS

This application is a National Stage Application of PCT application No. PCT/CN2023/125644, filed on Oct. 20, 2023, which claims the priority and benefit of Chinese patent applications No. 202211299473.7, filed on Oct. 21, 2022 and No. 202311315364.4, filed on Oct. 11, 2023. The entireties of PCT application No. PCT/CN2023/125644, Chinese patent applications No. 202211299473.7 and No. 202311315364.4 are hereby incorporated by reference herein and made a part of this specification.

### TECHNICAL FIELD

The present disclosure relates to the field of medicine, and in particular to a mucosal administration formulation and a preparation method and a use thereof.

### BACKGROUND ART

The mucosal system, including the mucosa of the oral cavity, nasal cavity, digestive tract, urethra, vagina, etc., is an important protective barrier on the human body and an important pathway for exogenous microorganisms to invade and drug molecules to enter the human body. Compared with the normal skin environment, the thickness of the mucosal layer is between 10-1000 micrometers. The mucosal layer has no keratinized surface and contains only a single layer or several layers of epithelial cells, which is conducive to the transmembrane transport of drug molecules. In addition, most mucosal surfaces contain microvilli, which further increase the surface area of the mucosa. Therefore, mucosa with high permeability and large surface area can serve as an important route for administration. Currently, there are many chemical small molecule drugs that can be administrated through the mucosa.

In another aspect, according to the principles of immunology, mucosal immunity can activate the expression of secretory antibodies sIgA on the mucosal surface, so as to directly block pathogens outside the body, fundamentally eliminate the occurrence of infection, and thus overcome the formation of asymptomatic infections under ordinary humoral/cellular immunity.

However, in the field of biomacromolecule drugs and nucleic acid drugs, there is currently no mature mucosal administration technology. The main difficulty is that biomacromolecules and negatively charged nucleic acids have difficulty penetrating cell membranes or cell intercellular spaces. Mucosal cells, especially micro-wrinkled cells with a large number of microvilli structures on their surface, tend to phagocytize large numbers of positively charged particles with a particle size between 100 nanometers to 1 micrometer. Therefore, transmucosal biological administration can be achieved by encapsulating biological macromolecules with such particles. The electrical property of nucleic acid is regulated by ion reaction, and the nucleic acid is coated with biocompatible materials to form nucleic acid nanoparticles with specific size, which can also realize transmucosal biological administration. Another difficulty to mucosal administration technology is the presence of a variety of nucleases on the mucosal surface, which affects the stability of nucleic acid drugs.

CN1812765B discloses a film-shaped dosage form for administering active ingredients to the human or animal body through the mucosa. During its preparation process, the pH value of the matrix used to prepare the dosage form for administration and including a solvent or a mixture of solvents, at least one polymer forming a matrix and at least one component selected from active pharmaceutical ingredients and aromatic substances is close to or adapted to the physiological pH value of the mucosa to which the dosage form for administration is applied. The use of this dosage form reduces or even prevents mucosal irritation during administration. The focus of this patent is on the “rapid release of active substances”. However, for peptide or protein drugs, their absorption process is generally slower than that of small molecule drugs, and the stability of the released peptide or protein drugs in the physiological environment of the mucosal surface is poor. Therefore, for peptide or protein drugs, sustained-release administration of nanoparticles has greater advantages than rapid-release administration of film formulations.

CN1720024 discloses a polymer foam or film composition for locally delivering pharmacologically effective agents to the nasal cavity, oral cavity, vagina, labia or scrotal epithelium or entering the systemic circulation through the nasal cavity, oral cavity, vagina, labia or scrotal epithelium. The composition contains at least one matrix polymer or a mixture of a matrix polymer and a pharmacologically effective agent. The composition further contains a penetration enhancer, an absorption enhancer, a mucoadhesive agent, a hydrophilic or hydrophobic release modifier, or a mixture thereof, and can be applied on the surface of a complex drug delivery system. The dosage form of this patent is foam or film, which has no nanoparticle structure. According to the literature, the mucosal layer has a high absorption capacity for nanoparticles of 100-1000 nm, so this nanoformulation administration system can be better applied to mucosal administration absorption. In addition, the penetration enhancer of this patent relies on “diol derivatives” (page 42, paragraph 15), while the preferred penetration enhancer of the disclosure does not contain diol derivatives, and the control example shows that the effect of the penetration enhancer selected by the disclosure is better than PEG400 (the diol derivative used in Example 1 of this patent).

CN1068585C and other similar patents disclose several cationic lipids for gene therapy. These lipids can encapsulate nucleic acid drugs through ion reactions, change their electrical properties, and ultimately allow nucleic acid drugs to enter cells in the form of nanoliposomes/lipid particles. However, such cationic materials often have certain cytotoxicity and their pharmaceutical applications are subject to many limitations. Moreover, liposomes/lipid particles themselves have the problem of aggregation during long-term storage, and their adsorption/permeability on the mucosal surface is insufficient.

CN108697803A discloses a transmucosal administrated drug composition, which includes a lipophilic active compound, a polymer matrix formed by two or more water-soluble polymers, and a fast dissolving agent. At least one of the water-soluble polymers is an amphiphilic polymer and at least one of the water-soluble polymers is a hydrophilic polymer or another amphiphilic polymer, and the hydrophilic-hydrophobic balance of this amphiphilic polymers is different from that of the first amphiphilic polymer. The applicable active ingredients of this patent are mainly “lipophilic active compounds”, but most protein and peptide drugs are not lipophilic and are therefore not applicable to the method of this patent.

In summary, the development of mucosal administration technology for active protein drugs and nucleic acid drugs is still inadequate, and more necessary research is needed on mucosal administrated formulations for nucleic acid drugs.

In view of this, the disclosure focuses on the administration of proteins, peptide drugs, and nucleic acid drugs, and therefore does not necessarily require the presence of amphiphilic polymers. According to the disclosure, hydrophilic polymers of pure natural origin can also achieve efficient mucosal administration of proteins, peptide drugs, and nucleic acid drugs. It can avoid the use of industrially synthesized amphiphilic polymer reagents and has better safety.

### SUMMARY

In view of the problems in the existing technology, the present disclosure provides a mucosal administration formulation and a preparation method and a use thereof, nanoparticles prepared by ionic crosslinking, and an application of mucosal administration with the nanoparticles.

The components of the nanoparticles include a nucleating agent, an active ingredient, a penetration enhancer, and a coating agent. The active ingredient is preferably a drug protein or a nucleic acid drug. Except for the active ingredients, the remaining components are all from the pharmaceutical excipients included in the “Chinese Pharmacopoeia (2020 Edition)”, and their safety has been fully verified.

The preparation principle is that the active ingredient forms agglomeration nuclei through electrostatic adsorption with the nucleating agent under specific pH conditions. By dispersing a low-concentration solution containing agglomerated nuclei in a solution containing a high-concentration coating agent, a nanoparticle solution containing the active ingredient can be quickly formed through ionic crosslinking between the nucleating agent and the coating agent. The size of the formed nanoparticles can be controlled by adjusting the concentration and the ionic conditions of the nucleating agent and the capping agent. By selecting a suitable nanoparticle solution, adding a penetration enhancer and then freeze-drying, a solid mucosal administration formulation containing nanoparticles can be obtained. It can achieve transmembrane absorption of active ingredients on mucosas at the oral cavity, nasal cavity, digestive tract, urethra, vagina, etc.

In order to achieve the above-mentioned purpose of the disclosure,

The present disclosure provides a preparation method of a mucosal administration formulation, including following steps:
(1) mixing a nucleating agent solution, an active ingredient solution, and an inorganic salt buffer solution to obtain a mixed solution;
(2) stirring a coating agent solution, spraying the coating agent solution into the mixed solution obtained in the step (1), adding a permeation enhancer, and freeze-drying to obtain the formulation;
   the active ingredient is selected from at least one of a drug protein and a nucleic acid drug;
   when the active ingredient is the drug protein, the nucleating agent or the coating agent comprises one or more of poloxamer 188, poloxamer 407, beta-cyclodextrin, hydroxypropyl beta-cyclodextrin, polyoxyl stearate, chitosan, carboxymethyl chitosan, carbomer 940p, alginic acid and its sodium salt, hyaluronic acid and its sodium salt, polyvinyl alcohol 2000, polyvinyl alcohol 4000, polyvinyl alcohol 6000, polyvinyl alcohol 8000, polysorbate 80, sodium carboxymethyl cellulose, sodium carboxymethyl starch, polycarbophil, and povidone;
   when the active ingredient is the nucleic acid drug, the nucleating agent or coating agent comprises one or more of poloxamer 188, poloxamer 407, beta-cyclodextrin, hydroxypropyl beta-cyclodextrin, polyoxyl stearate, polyglutamic acid, polyaspartic acid, polyarginine, polylysine, chitosan, carboxymethyl chitosan, alginic acid and its sodium salt, hyaluronic acid and its sodium salt, polyvinyl alcohol 2000, polyvinyl alcohol 4000, polyvinyl alcohol 6000, polyvinyl alcohol 8000, polysorbate 80, sodium carboxymethyl cellulose, sodium carboxymethyl starch, and povidone.

Preferably, when the active ingredient is the nucleic acid drug, the nucleating agent and the coating agent are not the same substance.

Preferably, when the active ingredient is the drug protein, the nucleating agent is alginic acid; when the active ingredient is the nucleic acid drug, the nucleating agent is chitosan or polylysine.

Preferably, when the active ingredient is the drug protein, a raw material of the coating agent is chitosan; when the active ingredient is the nucleic acid drug, the coating agent is alginic acid or dextran.

Further, the penetration enhancer comprises one or more of glucose, sucrose, maltose, trehalose, mannitol, sorbitol, EDTA and EGTA.

Preferably, when the active ingredient is the drug protein, the penetration enhancer is sucrose; when the active ingredient is the nucleic acid drug, the penetration enhancer is EDTA.

Preferably, the inorganic salt buffer solution is a phosphate buffer.

In the above steps, a stirring step may be included under certain conditions. For example, a stirring step may be selectively included when a mixing operation is involved. The mixing operation may be the mixing of two solutions or the mixing of a solution and a solid. According to general understanding, the preparation of a solution should also include a stirring step.

The above steps may also include a filtration and sterilization step well known in the art, which should be understood by those skilled in the art even if it is not limited. The solution is generally prepared with sterile water, but may also be prepared with other standard water.

Furthermore, when the active ingredient is the drug protein:

The concentration of the nucleating agent solution is a solution with a mass fraction of 0.01-5%, a pH range is 3-10. The ion concentration of the nucleating agent is 1-500 mM. Sterilization is carried out by filtration in the preparation process.

In some specific implementations, the nucleating agent solution is obtained by using phosphate buffer, sterile water and nucleating agent.

The concentration of the coating agent solution is a solution with a mass fraction of 0.1-30%, a pH range is 3-10. The ion concentration of the coating agent is 1-500 mM. Sterilization is carried out by filtration in the preparation process.

In some specific implementations, the coating agent solution is obtained by using acetic acid and coating agent.

In some specific implementations, the penetration enhancer solution is obtained by using penetration enhancer and sterile water.

The concentration range of the active ingredient solution is 1-100 mg/mL. A suitable phosphate buffer is selected for the drug protein solvent according to the properties of the active protein. Sterilization is carried out by filtration in the preparation process.

Furthermore, the drug protein solution is a COVID-19 antigen solution.

In some specific implementations, the production method of the mucosal administration formulation includes following steps:
infusing the nucleating agent solution, the COVID-19 antigen solution and the phosphate buffer into a liquid preparation tank in sequence, controlling the temperature at 4 °C, and mixing for 15 minutes.

Injecting the coating agent solution into the formulation tank, and spraying the liquid in the liquid preparation tank into the formulation tank through a sprayer.

Stirring is continued after spraying. Then dropping the penetration enhancer solution, and continuing stirring after dropping.

Dispensing the mixed solution in the formulation tank into penicillin bottles, 0.5 mL/bottle, with an automatic filling machine. Transferring the mixed solution after subpackaging into a lyophilizer and pre-freezing. Starting the lyophilization process. Sealing the lid after freeze-drying.

Taking samples and redissolving to test protein content, turbidity, viscosity, osmotic pressure, particle size and surface potential after the lyophilization is finished. The remaining samples are stored or tested for stability.

Furthermore, when the active ingredient is the nucleic acid drug:
the concentration of the nucleating agent solution is a solution with a mass fraction of 0.01%-5%, preferably 1%-3%, the pH range is 3-10, and the ion concentration of the nucleating agent is 1-500 mM.

In some specific implementations, the solvent of the nucleating agent solution is one or both of phosphate buffer and water.

The concentration of the coating agent solution is a solution with a mass fraction of 0.1%-20%, preferably 0.2%-10%, the pH range of 3-10, the ion concentration of the coating agent is 1-500 mM.

In some specific implementations, the solvent of the coating agent solution is water.

The penetration enhancer is added in the form of a penetration enhancer solution. In some embodiments, the solvent of the penetration enhancer solution is water. In some specific implementations, the penetration enhancer solution is an EDTA buffer solution with a pH range of 7-9, preferably 8-9, and a concentration of 400-600 mM, preferably 500-600 mM.

The concentration range of the active ingredient solution is 0.1-20 mg/mL, preferably 10-20 mg/mL.

The inorganic salt buffer is a phosphate buffer with a pH range of 4-7.

In the step (2), adding a lyophilization protectant at the same time by adding the penetration enhancer, the lyophilization protectant is added in a form of a solution. Preferably, the lyophilization protectant solution is a trehalose solution with a mass fraction of 20%-40%, more preferably 30%-40%, and even more preferably a trehalose solution with a mass fraction of 30%.

Preferably, the nucleic acid drug is antigen mRNA, and more preferably SARS-CoV-2 antigen mRNA.

In the present disclosure, the SARS-CoV-2 is sometimes also referred to as the novel coronavirus.

In some specific implementations, the preparation method of the mucosal administration formulation includes following steps:
1) injecting the phosphate buffer, the nucleating agent solution and the antigen mRNA solution into a low-temperature liquid preparation tank in sequence, controlling the temperature at 4 °C, and mixing for 15 minutes;
2) injecting the coating agent solution into the formulation tank, and spraying the liquid in the liquid preparation tank into the formulation tank through a sprayer at the rotating speed of 500-3000 rpm;
3) stirring is continued after spraying, then dropping the penetration enhancer solution and the lyophilization protectant, and continuing stirring after dropping;
4) dispensing the mixed solution in the formulation tank into penicillin bottles, 0.5 mL/bottle, with an automatic filling machine; transferring the mixed solution after subpackaging into a lyophilizer, pre-freezing; starting the lyophilization process; sealing the lid after freeze-drying.
5) taking samples and redissolving to test nucleic acid content, turbidity, viscosity, osmotic pressure, particle size and surface potential after the lyophilization is finished; the remaining samples are stored or tested for stability.

Preferably, the preparation method of the mucosal administration formulation includes following steps:
(1) mixing a nucleating agent solution, a SARS-CoV-2 antigen mRNA solution, and an inorganic salt buffer solution to obtain a mixed solution;
(2) spraying the mixed solution obtained in the step (1) into the coating agent solution, adding the penetration enhancer solution and a protective agent solution, stirring again, and freeze-drying to obtain the formulation;
in the step (1):
the volume ratio of the nucleating agent solution, the SARS-CoV-2 antigen mRNA solution and the inorganic salt buffer solution is 4-6:1-3:0.5-1.5, preferably 5:2:1;
in the step (2):
the volume ratio of the coating agent solution, the penetration enhancer solution and the protective agent solution is 8-10:1:1, preferably 10:1:1; and the volume ratio of the coating agent solution and the SARS-CoV-2 antigen mRNA solution in the step (1) is 4-6:1, preferably 5:1.

In some specific implementations, the preparation method includes following steps:
(1) mixing a chitosan solution, a SARS-CoV-2 antigen mRNA solution, and a phosphate buffer solution to obtain a mixed solution;
(2) spraying the mixed solution obtained in the step (1) into the alginic acid solution, adding the EDTA solution and the trehalose solution, stirring again, and freeze-drying to obtain the formulation;
in the step (1):
the volume ratio of the nucleating agent solution, the SARS-CoV-2 antigen mRNA solution and the phosphate buffer solution is 4-6:1-3:0.5-1.5, preferably 5:2:1;
in the step (2):
the volume ratio of the alginic acid solution, the EDTA solution and the trehalose solution is 8-10:1:1, preferably 10:1:1; and the volume ratio of the alginic acid solution and the SARS-CoV-2 antigen mRNA solution in step (1) is 4-6:1, preferably 5:1.

Furthermore, in the step (2), the freeze-drying comprises pre-freezing, main drying and desorbing; the pre-freezing time is 4-24 hours, the pre-freezing temperature is −80 °C, the main drying time is 4-72 hours, the main drying temperature is −80 °C to −10 °C and configured in gradient, the desorbing time is 2-24 hours, the desorbing temperature is −10 °C to 50 °C and configured in gradient.

Furthermore, the preparation of the mucosal administration formulation is completed in a Class A clean environment. A low-temperature liquid preparation tank and a formulation tank of a mechanical stirrer with 1-1000 spraying devices are used. The spray rate of each nozzle is 0.01-100 mL/min. The formulation temperature selection range is 0-50 °C.

In another aspect, the present disclosure also provides a mucosal administration formulation prepared by the above-mentioned preparation method.

Furthermore, the mucosal administration formulation is in the form of a solid dry powder with a water content between 0.5-10%. A nanoparticle solution is formed after the obtained formulation is redissolved in water. In particular, the particle size of the active component particles is 0.1-10 μM. The stability of the dry powder formulation is more than 1 year.

Furthermore, when the active ingredient is the drug protein, the water content of the mucosal administration formulation is between 1-10%, and when the active ingredient is the nucleic acid drug, the water content of the mucosal administration formulation is between 0.5%-5%.

The beneficial technical effects of the present disclosure are in that:

The present disclosure relates to a mucosal administration technology. The key point of this technology is to achieve transmembrane absorption of proteins, peptide drugs and nucleic acid drugs on the mucosa through drug-loaded nanoparticle. By optimizing the protein, the ability of the protein itself to target mucosal immune cells is enhanced. The polymer materials that comply with the pharmaceutical excipients of the “Chinese Pharmacopoeia” are used to form complexes with proteins, peptides, and nucleic acid drugs through electrostatic adsorption to optimize the structure, adhesion, and stability of the formulation. In addition, the activity of the loaded antigens and the mucosal absorption effect are improved. This vaccine program integrates nanotechnology with protein recombinant technology/nucleic acid drug technology. Through oral/nasal spray administration, it can innovatively and effectively induce mucosal, humoral, and cellular triple immunity pathways at the same time. It has the characteristics of high neutralizing antibody production efficiency, high safety, and room temperature stability.

### BRIEF DESCRIPTION OF THE DRAWINGS

FIG. 1 is a diagram showing the particle size distribution of the formulations of Example 1, Example 2 and Comparative Example 1.

FIG. 2 is a diagram showing the stability of the protein in the formulation of Example 1.

FIG. 3 is a comparison of cell absorption of the positive control, Examples 1-3, Control Example 1 and the negative control.

FIG. 4 is the neutralizing antibody titer on the mucosa after intramuscular injection, as well as mucosal administration in mice of Examples 1-3 and Control Example 1.

FIG. 5 is a particle size distribution diagram of drug-loaded nanoparticles of Examples 4-6 and Comparative Example 6.

FIG. 6 is a diagram showing the surface potential distribution of drug-loaded nanoparticles of Examples 4-6 and Comparative Example 6.

FIG. 7 is a diagram showing the cell absorption of drug-loaded nanoparticles of Examples 4-6 and Comparative Example 6.

FIG. 8 is the experimental effects of drug-loaded nanoparticle for mouse immunization of Examples 4-6 and Control Example 6.

### DETAILED DESCRIPTION

The following describes embodiment of the present disclosure through specific examples. Those skilled in the art can easily understand other advantages and effects of the present disclosure from the contents disclosed in this specification. The present disclosure may also be implemented or applied through other different specific implementation methods, and the details in this specification may also be modified or changed in various ways based on different viewpoints and applications without departing from the spirit of the present disclosure.

Before further describing the embodiment of the present disclosure, it should be understood that the scope of protection of the present disclosure is not limited to the specific embodiment described below. It should also be understood that the terms used in the examples of the present disclosure are for describing the specific embodiment rather than for limiting the scope of protection of the present disclosure.

When numerical ranges are given in the embodiment, it should be understood that both endpoints of each numerical range and any numerical value between the two endpoints can be selected unless otherwise specified in the present disclosure. Unless defined otherwise, all technical and scientific terms used herein have the same meaning as commonly understood by one of ordinary skill in the art to which this disclosure belongs.

It is worth noting that, the raw materials used in the present disclosure are all common commercially available products, so their sources are not specifically limited.

The sources of some raw materials in the present disclosure are as follows:

| Reagents | Brand/Manufacturer | Part Number |
|---|---|---|
| Chitosan | Sigma | 419419 |
| Alginic acid | Sigma | 180947 |
| Trehalose | Sangon Biotech | A600966 |
| Polylysine | Sigma | P1399 |
| Dextran | Sangon Biotech | A620219 |

### 1. Preparation of Active Protein Mucosal Administration Formulation

### Example 1

All liquid preparations were done in an isolator with sterile water to ensure the sterility of the solution. All reagents were placed on ice as far as possible to prevent degradation. The obtained working solution was packaged into appropriate specifications and frozen at −20 °C. Reagents at room temperature could only be used on the same day.

The phosphate buffer at pH 8, concentration 100 mM was prepared, filter sterilized or autoclaved.

The nucleating agent solution was prepared. A solution with 0.5% mass fraction of alginic acid was prepared with a phosphate buffer with a pH of 8 and sterile water. The salt concentration was 100 mM, and the volume was 200 mL. 100 mL of EDTA solution (0.5 M, pH=8) was added. Filter sterilization was performed after evenly mixed.

The coating agent solution was prepared. The chitosan was dissolved in 1% acetic acid. A final concentration was 1%. About 1 L was prepared, and sterilized by autoclave.

The concentration of the COVID-19 antigen solution was 5 mg/mL and filter sterilization was performed.

The penetration enhancer solution was prepared. 100 g of sucrose was weighed, dissolved in sterile water to prepare a 20% solution, and filter sterilization was performed.

Nanoparticle Preparation:

All operations were completed in a super-clean bench. The low-temperature liquid preparation tank, as well as the formulation tank of the mechanical stirrer with spraying devices were transferred to the isolator. The lyophilizer was turned on and the pre-freezing temperature was configured to −50 °C.

300 ml of the nucleating agent solution, 600 ml of the COVID-19 antigen solution and 100 ml of the phosphate buffer were infused into a liquid preparation tank in sequence. The temperature was controlled at 4 °C, and mixing time was 15 minutes.

1 L of the coating agent solution was infused into the formulation tank, and the rotation speed was configured to 800 rpm. The liquid in the liquid preparation tank was sprayed into the formulation tank through a sprayer at a spray rate of 50 mL/min. The nozzle height was configured to 30 cm.

Stirring was continued for 10 minutes after spraying. Then 400 mL of permeation enhancer was dropped at a dropping rate of 40 mL/min, and stirring was continued for 10 minutes after dropping.

Dispensing the mixed solution in the formulation tank into penicillin bottles, 0.5 mL/bottle, with an automatic filling machine. The mixed solution after subpackaging was transferred into a lyophilizer and pre-frozen for 4 h. The lyophilization process was started. The lid was sealed after freeze-drying.

taking samples and redissolving to test protein content, turbidity, viscosity, osmotic pressure, particle size and surface potential after the freeze-drying is finished. The remaining samples are stored or tested for stability.

### Example 2

The method was similar to that of Example 1, except that the nucleating agent was replaced with an equal amount of sodium hyaluronate.

### Example 3

The method was similar to that of Example 1, except that the coating agent was replaced with an equal amount of dextran.

### Comparative Example 1

The method was similar to that of Example 1, except that the coating agent was replaced with an equal amount of polyvinyl alcohol 400.

### 2. Preparation of Nucleic Acid Drug Mucosal Administration Formulation

### Example 4 Preparation Experiment of Nanoparticles

In this example, unless otherwise specified, all liquid preparations were done with sterile water as a solvent in an isolator to ensure that the solutions were sterile. All reagents were placed on ice as far as possible to prevent degradation. The obtained working solution was packaged into appropriate specifications and frozen at −20 °C. Reagents at room temperature could only be used on the same day.

### (1) Preparation of Basic Solution:

The phosphate buffer at pH 5, concentration 100 mM was prepared, filter sterilized or autoclaved.

The nucleating agent solution was prepared. A solution with 1% mass fraction of chitosan was prepared with a phosphate buffer with a pH of 5 and sterile water. The phosphate concentration was 10 mM, and the volume was 200 mL. Filter sterilization was performed after fully dissolved evenly mixed.

The coating agent solution was prepared. The alginic acid was dissolved in sterile water. A final concentration was 0.2%. About 1 L was prepared, and sterilized by autoclave.

The concentration of the COVID-19 antigen mRNA solution was 20 mg/mL and filter sterilization was performed.

The EDTA buffer at pH 8, concentration 500 mM was prepared, filter sterilized or autoclaved.

The lyophilization protectant solution was prepared. 100 g of trehalose was weighed, dissolved in sterile water to prepare a solution with a mass fraction of 30%, and filter sterilization was performed.

### (2) Nanoparticle Preparation:

All operations were completed in a super-clean bench. The low-temperature liquid preparation tank, as well as the formulation tank of the mechanical stirrer with spraying devices were transferred to the isolator. The lyophilizer was turned on and the pre-freezing temperature was configured to −50 °C.

500 ml of the nucleating agent solution, 200 ml of the COVID-19 antigen solution and 100 ml of the phosphate buffer were infused into a liquid preparation tank in sequence. The temperature was controlled at 4 °C, and mixing time was 15 minutes.

1 L of the coating agent solution was infused into the formulation tank, and the rotation speed was configured to 1500 rpm. The liquid in the liquid preparation tank was sprayed into the formulation tank through a sprayer at a spray rate of 120 mL/min. The nozzle height was configured to 30 cm.

Stirring was continued for 10 minutes after spraying. Then 100 mL of permeation enhancer was dropped at a dropping rate of 10 mL/min, and 100 mL of lyophilization protectant was continued to drop at a dropping rate of 50 mL/min, and stirring was continued for 10 minutes after dropping.

Dispensing the mixed solution in the formulation tank into penicillin bottles, 0.5 mL/bottle, with an automatic filling machine. The mixed solution after subpackaging was transferred into a lyophilizer and pre-freezing for 4 h. The freeze-drying process was started. sealing the lid after freeze-drying.

Samples were taken and redissolved to test nucleic acid content, turbidity, viscosity, osmotic pressure, particle size and surface potential after the freeze-drying is finished. The remaining samples are stored or tested for stability.

### Example 5

Referring to Example 1, the coating agent was replaced by dextran instead of alginic acid.

### Example 6

Referring to Example 4, the nucleating agent was replaced by polylysine instead of chitosan.

### Comparative Example 2

Referring to Example 4, except that both the coating agent and the nucleating agent are alginic acid.

### Comparative Example 3

Referring to Example 4, except that both the coating agent and the nucleating agent are chitosan.

### Comparative Example 4

Referring to Example 4, except that both the coating agent and the nucleating agent are dextran.

### Comparative Example 5

Referring to Example 4, except that both the coating agent and the nucleating agent are polylysine.

### Comparative Example 6

The preparation was performed according to the method of Example 17 of CN102625696B. The COVID-19 antigen solution was used instead of the siRNA in this example.

### Comparative Example 7

The drug formulation was prepared with reference to the preparation method of Example 1 in CN115590826A, with the difference that the COVID-19 antigen solution (protein drug) was replaced by the COVID-19 antigen mRNA solution (nucleic acid drug) of Example 1 of the present disclosure.

### 3. Characterization of Nanoparticles

Preferably, the particle size and surface potential of the nanoparticles were evaluated using a Zetasizer particle size analyzer from Malvern. The test could also be completed using other instruments with similar test principles and test capabilities.

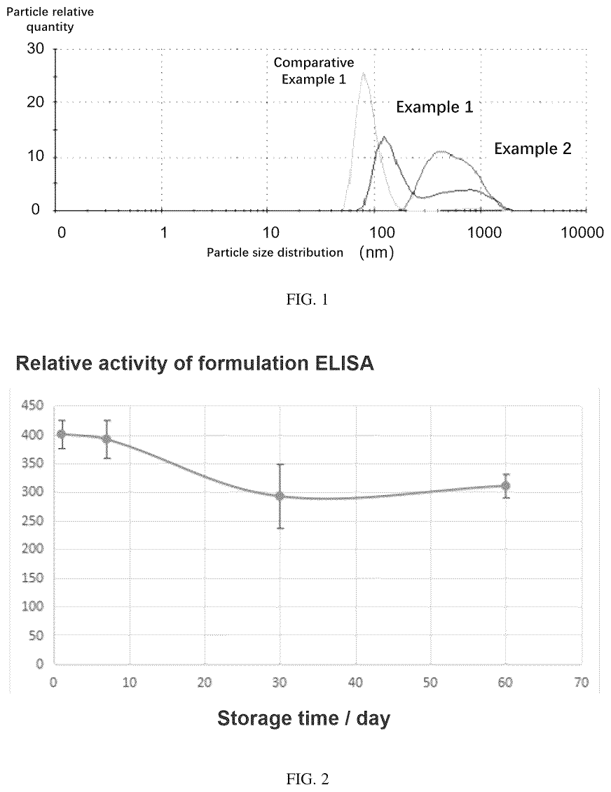

One formulation product was randomly taken and infused 0.5 mL of water for injection. The upside-down shaking was performed for 10 times. The solution was extracted and mixed with 0.5 mL of the phosphate buffer with a specific pH, preferably 20 mM phosphate buffer with pH 7.4 in the present disclosure. It was placed in sample cell and the particle size was measured. The measurements were performed three times in succession and the values were averaged. The particle size of the nanoparticles in Example 1 was between 0.3-1 μM. Relevant experiments of the products of Examples 1-2 and Comparative Example 1 were carried out simultaneously, and the particle size distribution diagram of the obtained drug-loaded nanoparticles is shown in FIG. 1. The particle sizes of the nanoparticles in Examples 4-6 were between 255.97-585.97 nm, which was consistent with the trend particle size of 100 nm to 600 nm. FIG. 5 shows the particle size distribution diagram of drug-loaded nanoparticles of Examples 4-6 and Comparative Example 6.

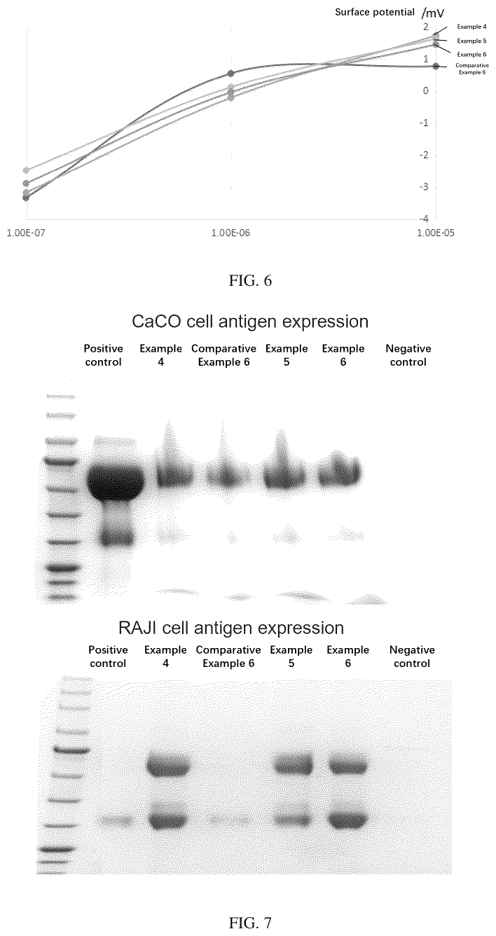

10 formulation products were randomly taken and each product was infused 0.5 mL of water for injection. The upside-down shaking was performed for 10 times. The solution was extracted and mixed with 0.5 mL of the phosphate buffer with continuously changing pH respectively, preferably 20 mM phosphate buffer with pH of 6.0, 6.2, 6.4, 6.6, 6.8, 7.0, 7.2, 7.4, 7.6, and 7.8 in this example. According to the pH sequence, each sample was placed in the sample cell in sequence and the surface potential was measured. The measurements were performed three times in succession and the values were averaged. The resulting surface potential was plotted against pH, from which the isoelectric point of the nanoparticles and the surface potential under physiological conditions were read. FIG. 6 shows a diagram showing the surface potential distribution of drug-loaded nanoparticles of Examples 4-6 and Comparative Example 5.

The specific results of Examples 4-6 and Comparative Examples 2-7 were as follows:

| | Nanoparticle | Isoelectric | Surface |
|---|---|---|---|
| Group | size nM | point | potential mV |
| Example 4: | 585.97 | 6.0 | −3.34 |
| Example 5: | 338.63 | 6.5 | −2.18 |
| Example 6: | 255.97 | 6.1 | −2.48 |
| Comparative | 941.99 | 4.9 | −4.05 |
| Example 2 | | | |
| Comparative | 945.33 | 6.5 | −2.25 |
| Example 3 | | | |
| Comparative | 758.53 | 7.0 | 0.11 |
| Example 4 | | | |
| Comparative | 651.63 | 8.6 | 5.69 |
| Example 5 | | | |
| Comparative | 88.05 | 7.4 | 4.85 |
| Example 6 | | | |
| Comparative | 1716.58 | 7.5 | 4.42 |
| Example 7 | | | |

### 2. Evaluation of Drug-Loaded Nanoparticle Protein

One formulation product was randomly taken and infused 0.5 mL of water for injection. The upside-down shaking was performed for 10 times. The solution was extracted and centrifuged with an ultra-high speed centrifuge at 100,000 rpm, 4 °C, for 24 hours. The supernatant was aspirated. The protein content was determined by UV spectrophotometry. For the formulation in Example 1, the protein content in the supernatant was not higher than 0.025 mg/mL. That is, the protein encapsulation efficiency is greater than 96%. The specific results of Examples 4-6 and Comparative Examples 2-7 were as follows:

| | | Nucleic acid |
|---|---|---|
| | Nucleic acid content in | encapsulation |
| Group | supernatant mg/mL | efficiency % |
| Example 4: | 0.08 | 97 |
| Example 5: | 0.22 | 89 |
| Example 6: | 0.16 | 92 |
| Comparative | 1.76 | 12 |
| Example 2 | | |
| Comparative | 1.12 | 44 |
| Example 3 | | |
| Comparative | 1.42 | 29 |
| Example 4 | | |
| Comparative | 0.84 | 58 |
| Example 5 | | |
| Comparative | 0.08 | 96 |
| Example 6 | | |
| Comparative | 1.22 | 39 |
| Example 7 | | |

### 3. Evaluation of Nanoparticle Protein Stability

This experiment was completed using commercial ELISA detection kits and SDS-PAGE kits.

Ten formulation products of Example 1 were randomly selected and stored at 4 °C. They were taken out at 0 day, 10 days, 20 days, 30 days, 50 days, 100 days, 150 days, 200 days, 300 days, and 365 days, respectively. 0.5 mL of water for injection was infused immediately thereafter. The upside-down shaking was performed for 10 times. The solution was extracted and tested using PAGE and ELISA tests under non-reducing conditions. The nucleic acids in the samples were extracted using a commercial nucleic acid extraction kit (Suzhou Beaver #70410), and the nucleic acid integrity was determined using a capillary electrophoresis kit (Hangzhou Houze Qsep100). ELISA test showed that the total preservation rate of the lyophilized and reconstituted products of Examples 1-3 was always above 80% compared with that of Control Example 1. Further analysis of the total stored protein using non-reducing PAGE showed that the protein with intact molecular weight was always above 95%, which indicated that the solid formulation had good stability at 4 °C within the shelf life of 1 year. The results of nucleic acid integrity showed that the total preservation rate (%) of lyophilized and reconstituted products was always at a higher level compared with the control group. This indicated that the solid formulation had good stability at 4 °C within a shelf life of 1 year.

The stability diagram of the drug-loaded nanoparticles of the present disclosure is shown in FIG. 2. The specific results of the preservation rate (%) of Examples 4-6 and Comparative Examples 2-7 were as follows:

| | 0 | 10 | 20 | 30 | 50 | 100 | 200 | 300 | 365 |
|---|---|---|---|---|---|---|---|---|---|
| Group | days | days | days | days | days | days | days | days | days |
| Example 4: | 100 | 100 | 100 | 101 | 99 | 99 | 97 | 95 | 95 |
| Example 5: | 100 | 100 | 100 | 98 | 95 | 90 | 65 | 35 | 15 |
| Example 6: | 100 | 100 | 101 | 100 | 99 | 95 | 83 | 71 | 65 |
| Comparative | 100 | 44 | 20 | 9 | 0 | 0 | 0 | 0 | 0 |
| Example 2 | | | | | | | | | |
| Comparative | 100 | 49 | 22 | 7 | 0 | 0 | 0 | 0 | 0 |
| Example 3 | | | | | | | | | |
| Comparative | 100 | 46 | 20 | 8 | 0 | 0 | 0 | 0 | 0 |
| Example 4 | | | | | | | | | |
| Comparative | 100 | 51 | 25 | 5 | 0 | 0 | 0 | 0 | 0 |
| Example 5 | | | | | | | | | |
| Comparative | 100 | 100 | 100 | 95 | 86 | 65 | 15 | 0 | 0 |
| Example 6 | | | | | | | | | |
| Comparative | 100 | 89 | 77 | 62 | 39 | 13 | 0 | 0 | 0 |
| Example 7 | | | | | | | | | |

### 4. Evaluation of Cellular Absorption of Drug-Loaded Nanoparticles

Unless otherwise stated, all chemicals were purchased from Sigma-Aldrich (St. Louis, MO). Transwell polycarbonate filter inserts (12-well, 3 mm pore size) were purchased from Corning Costar (New York, NY).

### (1) Measurement of Active Protein Mucosal Administration Formulation

Caco-2 cells were cultured in DMEM (25 mM glucose) with 10% (v/v) FBS (Hyclone Laboratories, Logan, UT), and Raji cells were cultured in RPMI 1640 medium supplemented with 10% (v/v) FBS.

0.1 and 0.5 mL of DMEM supplemented with 10% FBS were added to the top and basolateral sides of the cross-wells, respectively, and then the cross-wells were pre-incubated in a CO2 incubator for 30 minutes. Caco-2 cells were then spread to the top of the transwell. After 3 h of incubation, the medium on the top side was replaced, and the upper medium was replaced every other day for the next 14 d of culture. Raji cells suspended in a mixture of RPMI 1640/DMEM (1:2) were then added to the basolateral chamber of the cross-wells, and co-culture was maintained for 7 days.

One formulation product was randomly taken and infused 0.5 mL of water for injection, and the upside-down shaking was performed for 10 times. The solution was extracted for 100 μL to incubate on the top of the transwell. 100 μL of saline sample containing 125 μg of protein was added to the control well, and 100 μL of saline was added to the negative control well. The cross-wells were then pre-incubated in a CO₂ incubator for 2 h. The culture medium and caco2 cells were collected and assayed. SDS-PAGE was tested. It can be observed that in the caco2 cell recovery samples, obvious target protein signals were only observed in the formulation group. It was judged that the absorption effect was more than 50% compared with the standard concentration sample.

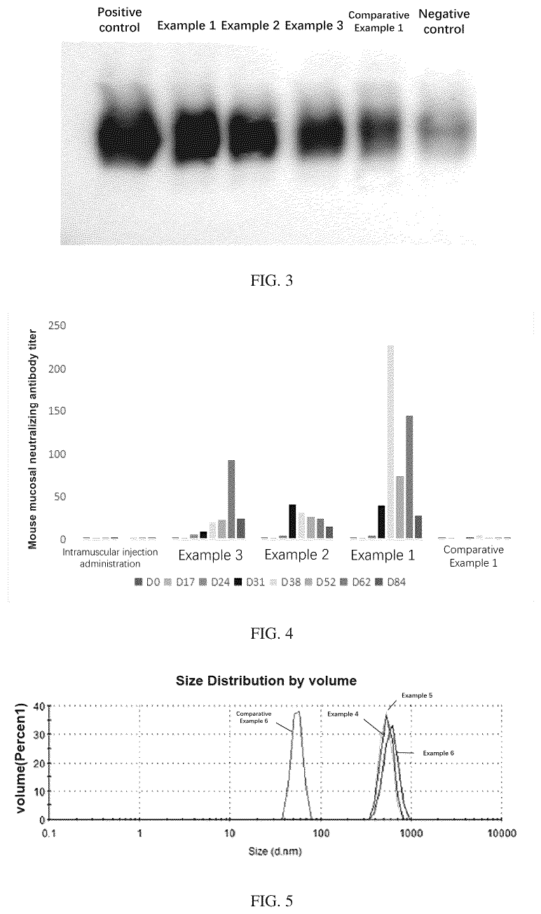

Relevant experiments of the products of Examples 1-3 and Comparative Example 1 were carried out simultaneously, and the cell absorption diagram of the obtained drug-loaded nanoparticles is shown in FIG. 3.

### (2) Measurement of Nucleic Acid Drug Mucosal Administration Formulation

Caco-2 cells (ATCC #HTB-37) were cultured in DMEM (25 mM glucose) with 10% (v/v) FBS (Hyclone Laboratories, Logan, UT), and Raji cells (ATCC #CCL-86) were cultured in RPMI 1640 medium supplemented with 10% (v/v) FBS.

0.1 ml and 0.5 mL of DMEM supplemented with 10% FBS were added to the top and basolateral sides of the cross-wells, respectively, and then the cross-wells were pre-incubated in a CO₂ incubator for 30 minutes. Caco-2 cells were then spread to the top of the transwell. After 3 h of incubation, the medium on the top side was replaced, and the upper medium was replaced every other day for the next 14 d of culture. Raji cells suspended in a mixture of RPMI 1640/DMEM (1:2) were then added to the basolateral chamber of the cross-wells, and co-culture was maintained for 7 days.

One formulation product was randomly taken and infused 0.5 mL of water for injection, and the upside-down shaking was performed for 10 times. The solution was extracted for 100 μL to incubate on the top of the transwell. 200 μg of COVID-19 antigen mRNA and appropriate liposome transfection reagent, such as lipofectine, were added to the positive control well, and diluted to 100 μL by injection with water, and 100 μL of saline was added to the negative control well. The cross-wells were then pre-incubated in a CO₂ incubator for 2 h. The reagents in the wells were replaced with normal culture medium and the culture was continued for 48 hours. The upper layer of Caco-2 cells and the lower layer of Raji cells were harvested and assayed. SDS-PAGE was tested. It can be observed that in the Caco-2 cell recovery samples, obvious target protein signals were observed in the formulation group and the positive group. In Raji cells, target protein signals were observed only in the formulation group. It was judged that the absorption effect was significantly better than that of the liposome transfection scheme compared with the positive group standard nucleic acid-lipofectine group samples. FIG. 7 shows a diagram showing the cell absorption of drug-loaded nanoparticles of Examples 4-6 and Comparative Example 6.

### 5. Animal Model Evaluation of Drug-Loaded Nanoparticles

### (1) Measurement of Active Protein Mucosal Administration Formulation

SPF BALB/c mice, aged 6-8 weeks and weighing about 20 g, were selected. These mice were raised in a clean environment.

The mice were randomly divided into 3 groups, with 10 mice in each group, half of them were male and half were female. After enrollment, 10 μL of blood was collected every week for blood routine testing for 3 weeks to confirm that there was no abnormality.

The administration experiment was carried out at the first, third and seventh weeks. One formulation product was randomly taken by each administration, and infused 0.5 mL of water for injection, and the upside-down shaking was performed for 10 times. The solution was extracted. In the formulation group, 25 μL of the formulation solution was sprayed into each nostril of each mouse. In the protein control group, saline solution containing 0.625 mg/mL antigen protein was used, and 25 μL was sprayed into each nostril of each mouse. In the negative control group, 25 μL of saline was sprayed into each nostril of each mouse.

The breathing rate of the mice was observed before and during each spraying and counted with a stopwatch. The respiratory rate was recorded. Before and after each spraying, the mice were weighed respectively. Their weight data were recorded, and their body temperature were measured.

After enrollment, before each administration experiment, and 1 week, 2 weeks, 3 weeks, 4 weeks, 2 months, 3 months, and 6 months after the third administration, mucosal sampling and venous blood collection were performed on mice. 100 μL of venous blood was drawn from mice each time, anticoagulated with 0.15 mg of EDTA-K2, and 10 μL was taken for analysis by a routine blood analyzer, and the data was recorded. The remaining blood samples were centrifuged at 3000 rpm for 20 min at 4 °C, and 20 μL of the supernatant plasma was immediately used for ELISA activity test to test the antibody level against the administered protein in mice. The remaining plasma was directly frozen in liquid nitrogen. Oral swabs were used to collect mucosal samples from mice. The obtained samples were immediately placed in protein preservation solution, and 20 μL was immediately used for ELISA activity test to test the antibody level against antigen protein on the mouse mucosa.

On the day after the third administration of the mice, half of the mice in each group were executed, and their main organs were collected and fixed with formaldehyde. Paraffin sections were made from the mouse nasal cavity and lung tissues, followed by routine dewaxing, hydration, stained with hematoxylin and eosin, dehydrated with gradient alcohol, transparentized with xylene, and sealed with neutral gum. The images were taken under a high-power microscope and the acute injury score was calculated with the Smith method.

The results obtained show that the formulation of the present disclosure can effectively induce immune activity against protein antigens and has no obvious irritation to mice.

Relevant experiments of the products of Examples 1-3 and Comparative Example 1 were carried out simultaneously, and the experimental effect of the obtained drug-loaded nanoparticles for mouse immunization is shown in FIG. 4.

### (2) Measurement of Nucleic Acid Drug Mucosal Administration Formulation

The pharmaceutical stability of Control Examples 2-5 and Control Example 7 is insufficient, and it is not meaningful to conduct antibody evaluation at the animal level. This evaluation experiment only verifies Examples 4-6 and Control Example 6.

SPF BALB/c mice, aged 6-8 weeks and weighing about 20 g, were selected. These mice were raised in a clean environment.

The mice were randomly divided into 3 groups, with 10 mice in each group, half of them were male and half were female. After enrollment, 10 μL of blood was collected every week for blood routine testing for 3 weeks to confirm that there was no abnormality.

The administration experiment was carried out at the first, third and seventh weeks. One formulation product was randomly taken by each administration, and infused 0.5 mL of water for injection, and the upside-down shaking was performed for 10 times. The solution was extracted. In the formulation group, 25 μL of the formulation solution was sprayed into each nostril of each mouse. In the antigen control group, saline solution containing 2 mg/mL COVID-19 mRNA was used, and 25 μL was sprayed into each nostril of each mouse. In the negative control group, 25 μL of saline was sprayed into each nostril of each mouse.

The breathing rate of the mice was observed before and during each spraying and counted with a stopwatch. The respiratory rate was recorded. Before and after each spraying, the mice were weighed respectively. Their weight data were recorded, and their body temperature were measured.

After enrollment, before each administration experiment, and 1 week, 2 weeks, 3 weeks, 4 weeks, 2 months, 3 months, and 6 months after the third administration, mucosal sampling and venous blood collection were performed on mice. 100 μL of venous blood was drawn from mice each time, anticoagulated with 0.15 mg of EDTA-K2, and 10 μL was taken for flow cytometric analysis to determine the proportion of CD8+ cells. The remaining blood samples were centrifuged at 3000 rpm for 20 min at 4 °C, and 20 μL of the supernatant plasma was immediately used for ELISA activity test (Abace Biotechnoligy, COVID-19 IgG Antibody Detection Kit (Enzyme-Linked Immunoassay)) to test the antibody level against the administered protein in mice. The remaining plasma was directly frozen in liquid nitrogen. Oral swabs were used to collect mucosal samples from mice. The obtained samples were immediately placed in protein preservation solution, and 20 μL was immediately used for ELISA activity test (Abace Biotechnoligy, COVID-19 IgA Antibody Detection Kit (Enzyme-Linked Immunoassay)) to test the antibody level against antigen protein on the mouse mucosa. The geometric mean titer (GMT) is a commonly used indicator to measure the viral antibody titer, which is the geometric mean of the viral antibody titers in all tested samples. The results were as follows:

| Group | Antibody level (GMT) |
|---|---|
| Example 4: | 434.524 |
| Example 5: | 100.107 |
| Example 6: | 166.723 |
| Comparative Example 6 | 29.854 |

On the day after the third administration of the mice, half of the mice in each group were executed, and their main organs were collected and fixed with formaldehyde. Paraffin sections were made from the mouse nasal cavity and lung tissues, followed by routine dewaxing, hydration, stained with hematoxylin and eosin, dehydrated with gradient alcohol, transparentized with xylene, and sealed with neutral gum. The images were taken under a high-power microscope and the acute injury score was calculated with the Smith method. The results were as follows:

| Group | Smith score |
|---|---|
| Example 4: | 0 |
| Example 5: | 0 |
| Example 6: | 1 |
| Comparative Example 6 | 1 |

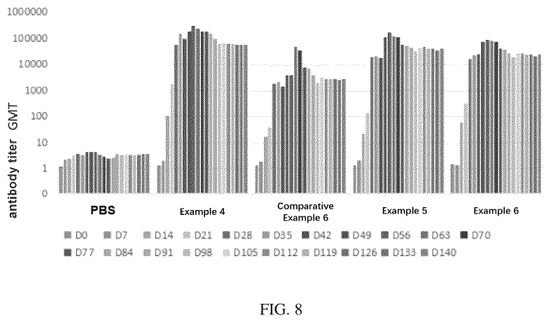

FIG. 8 shows the experimental effects of drug-loaded nanoparticle for mouse immunization of Examples 4-6 and Control Example 6 (GMT titer of each antibody).

The results obtained show that the formulation of the present disclosure can effectively induce immune activity against protein antigens and has no obvious irritation to mice.

It should be noted that the above content is only used to illustrate the present disclosure, rather than to limit the scope of protection of the present disclosure. Simple modifications or equivalent substitutions of the present disclosure by ordinary skilled in the art do not deviate from the essence and scope of the present disclosure.

1. A preparation method of mucosal administration formulation, comprising following steps:
(1) mixing a nucleating agent solution, an active ingredient solution, and an inorganic salt buffer solution to obtain a mixed solution;
(2) stirring a coating agent solution, spraying the coating agent solution into the mixed solution obtained in the step (1), adding a permeation enhancer, and freeze-drying to obtain the formulation;
the active ingredient is selected from at least one of a drug protein and a nucleic acid drug;
when the active ingredient is the drug protein, the nucleating agent or the coating agent comprises one or more of poloxamer 188, poloxamer 407, beta-cyclodextrin, hydroxypropyl beta-cyclodextrin, polyoxyl stearate, chitosan, carboxymethyl chitosan, carbomer 940p, alginic acid and its sodium salt, hyaluronic acid and its sodium salt, polyvinyl alcohol 2000, polyvinyl alcohol 4000, polyvinyl alcohol 6000, polyvinyl alcohol 8000, polysorbate 80, sodium carboxymethyl cellulose, sodium carboxymethyl starch, polycarbophil, and povidone;
when the active ingredient is the nucleic acid drug, the nucleating agent or coating agent comprises one or more of poloxamer 188, poloxamer 407, beta-cyclodextrin, hydroxypropyl beta-cyclodextrin, polyoxyl stearate, polyglutamic acid, polyaspartic acid, polyarginine, polylysine, chitosan, carboxymethyl chitosan, alginic acid and its sodium salt, hyaluronic acid and its sodium salt, polyvinyl alcohol 2000, polyvinyl alcohol 4000, polyvinyl alcohol 6000, polyvinyl alcohol 8000, polysorbate 80, sodium carboxymethyl cellulose, sodium carboxymethyl starch, and povidone.

2. The preparation method according to claim 1, wherein when the active ingredient is the nucleic acid drug, the nucleating agent and the coating agent are not the same substance.

3. The preparation method according to claim 1, wherein when the active ingredient is the drug protein, the nucleating agent is alginic acid; when the active ingredient is the nucleic acid drug, the nucleating agent is chitosan or polylysine.

4. The preparation method according to claim 1, wherein when the active ingredient is the drug protein, a raw material of the coating agent is chitosan; when the active ingredient is the nucleic acid drug, the coating agent is alginic acid or dextran.

5. The preparation method according to claim 1, wherein the penetration enhancer comprises one or more of glucose, sucrose, maltose, trehalose, mannitol, sorbitol, EDTA and EGTA.

6. The preparation method according to claim 5, wherein when the active ingredient is the drug protein, the penetration enhancer is sucrose; when the active ingredient is the nucleic acid drug, the penetration enhancer is EDTA.

7. The preparation method according to claim 1, wherein the inorganic salt buffer solution is a phosphate buffer.

8. The preparation method according to claim 1, wherein when the active ingredient is the drug protein:
a concentration of the nucleating agent solution is a solution with a mass fraction of 0.01-5%, a pH range is 3-10, and an ion concentration of the nucleating agent is 1-500 mM.

9. The preparation method according to claim 1, wherein when the active ingredient is the drug protein: a concentration of the coating agent solution is a solution with a mass fraction of 0.1-30%, a pH range is 3-10, and an ion concentration of the coating agent is 1-500 mM.

10. The preparation method according to claim 1, wherein when the active ingredient is the drug protein: a concentration range of the active ingredient solution is 1-100 mg/mL.

11. The preparation method according to claim 1, wherein when the active ingredient is the drug protein: the drug protein solution is a COVID-19 antigen solution.

12. The preparation method according to claim 1, wherein when the active ingredient is the nucleic acid drug:
a concentration of the nucleating agent solution is a solution with a mass fraction of 0.01%-5%, preferably 1%-3%, a pH range is 3-10, an ion concentration of the nucleating agent is 1-500 mM, a solvent of the nucleating agent solution is one or both of phosphate buffer and water.

13. The preparation method according to claim 1, wherein when the active ingredient is the nucleic acid drug:
a concentration of the coating agent solution is a solution with a mass fraction of 0.1%-20%, preferably 0.2%-10%, a pH range of 3-10, an ion concentration of the coating agent is 1-500 mM, a solvent of the coating agent solution is water.

14. The preparation method according to claim 1, wherein when the active ingredient is the nucleic acid drug:
the penetration enhancer is added in a form of a penetration enhancer solution, the penetration enhancer solution is an EDTA buffer solution with a pH range of 7-9 and a concentration of 400-600 mM.

15. The preparation method according to claim 1, wherein when the active ingredient is the nucleic acid drug:
a concentration range of the active ingredient solution is 0.1-20 mg/mL.

16. The preparation method according to claim 1, wherein when the active ingredient is the nucleic acid drug:
the inorganic salt buffer is a phosphate buffer with a pH range of 4-7.

17. The preparation method according to claim 1, wherein when the active ingredient is the nucleic acid drug:
in the step (2), adding a lyophilization protectant at the same time by adding the penetration enhancer, the lyophilization protectant is added in a form of a solution.

18. The preparation method according to claim 17, wherein the lyophilization protectant solution is a trehalose solution with a mass fraction of 20%-40%.

19. The preparation method according to claim 1, wherein when the active ingredient is the nucleic acid drug: the nucleic acid drug is antigen mRNA.

20. The preparation method according to claim 1, wherein the preparation method of the mucosal administration formulation comprises following steps:
(1) mixing a nucleating agent solution, a SARS-CoV-2 antigen mRNA solution, and an inorganic salt buffer solution to obtain a mixed solution;
(2) spraying the mixed solution obtained in the step (1) into the coating agent solution, adding the penetration enhancer solution and a protective agent solution, stirring again, and freeze-drying to obtain the formulation.

21. The preparation method according to claim 20, wherein in the step (1), a volume ratio of the nucleating agent solution, the SARS-CoV-2 antigen mRNA solution and the inorganic salt buffer solution is 4-6:1-3:0.5-1.5; in the step (2), a volume ratio of the coating agent solution, the penetration enhancer solution and the protective agent solution is 8-10:1:1, and a volume ratio of the coating agent solution and the SARS-CoV-2 antigen mRNA solution in the step (1) is 4-6:1.

22. The preparation method according to claim 20, wherein in the step (1), a volume ratio of the nucleating agent solution, the SARS-CoV-2 antigen mRNA solution and the inorganic salt buffer solution is 52:1; in the step (2), a volume ratio of the coating agent solution, the penetration enhancer solution and the protective agent solution is 10:1:1, and a volume ratio of the coating agent solution and the SARS-CoV-2 antigen mRNA solution in the step (1) is 5:1.

23. The preparation method according to claim 1, wherein in the step (2), the freeze-drying comprises pre-freezing, main drying and desorbing; the pre-freezing time is 4-24 hours, the pre-freezing temperature is −80 °C, the main drying time is 4-72 hours, the main drying temperature is −80 °C to −10 °C and configured in gradient, the desorbing time is 2-24 hours, the desorbing temperature is −10 °C to 50 °C and configured in gradient.

24. A mucosal administration formulation prepared by the preparation method according to claim 1.
